(19) United States
(12) Patent Application Publication
(10) Pub. No.: US 2011/0066104 A1
(43) Pub. Date: Mar. 17, 2011

(54) DEVICE FOR NEEDLELESS TRANSDERMAL DELIVERY OF MEDICATION

(75) Inventors: Giovanni Nisato, Eindhoven (NL); Roelf Kassies, Eindhoven (NL); Wilhelmus Henrikus Nicolaas Maria Van Mensvoort, Eindhoven (NL)

(73) Assignee: KONINKLIJKE PHILIPS ELECTRONICS N.V.

(21) Appl. No.: 12/992,283

(22) PCT Filed: May 15, 2009
(86) PCT No.: PCT/IB09/52029

§ 371 Date: Nov. 12, 2010

(30) Foreign Application Priority Data
May 20, 2008 (EP) 08156519.4

(51) Int. Cl. A61M 5/30

(52) U.S. Cl. 604/68

(57) ABSTRACT

The invention relates to a device for needleless transdermal drug delivery. The device according to the invention comprises an actuator (104) and a support (102) which has a support stiffness. The actuator (104) has a driver part (108) and a support part (106), which support part (106) is connected to the support and which driver part (108) is displaceable with regard to the support part (106) along an axis of displacement (110). The device according to the invention is characterized in that the support stiffness provides a substantially zero displacement of the actuator's support part (106) along the driver's axis of displacement (110) during a displacement of the driver part (108) along said axis with regard to the support part (106).

### FIELD OF THE INVENTION

The invention relates to a device for needleless transdermal delivery of medication, comprising an actuator and a support, which support has a support stiffness and which actuator has a driver part and a support part, which support part is connected to the support and which driver part is displaceable with regard to the support part along an axis of displacement. The invention furthermore relates to a cartridge for use in the device for needleless transdermal delivery of medication.

### BACKGROUND OF THE INVENTION

Techniques disclosed in U.S. Pat. No. 5,840,062 provide an apparatus and corresponding method for the rapid delivery of a fluid. The known apparatus comprises at least one first housing having walls defining a dispensing chamber for containing the fluid to be dispensed, at least one nozzle element defined in at least one of said walls for the discharge there through of said fluid. The apparatus further comprises a second housing which is adapted for detachable association to said first housing, including pump means for forcing a predetermined volume of said fluid out of said dispensing chamber and through said nozzle element. The pump means comprises at least one piezoelectric element adapted to cooperate with said first housing to impose a predetermined pressure within said dispensing chamber to force the predetermined volume of said fluid through the nozzle element and to deliver said fluid to the intended target.

Techniques disclosed in U.S. Pat. No. 5,840,062 eventuate in an inaccurate pressure in the dispensing chamber after cooperation with the piezoelectric element.

### OBJECT OF THE INVENTION It is an object of the invention to provide a device in which an accurate force can be generated for needleless transdermal delivery of medication.

### SUMMARY OF THE INVENTION

The object of the invention is achieved by the device according to the invention which is characterized in that the support stiffness provides a substantially zero displacement of the actuator's support part along the axis of displacement during a displacement of the driver part along said axis with regard to the support part. Here, a substantially zero displacement of the actuator's support part along the axis of displacement during a displacement of the driver part along said axis with regard to the support part denotes that a support's part displacement along the axis of displacement is substantially less, i.e. at least a factor 100, than a driver's part displacement along the axis of displacement.

In this text, a stiffness means a resistance of a deformable body to deformation through an applied force or torque. The support stiffness is to be understood as the stiffness of a construction of the support.

As mentioned before, the support stiffness provides a substantially zero displacement of the actuator's support part along the axis of displacement during a displacement of the driver part along said axis with regard to the support part. By annulling the displacement for the actuator's support part, the displacement of the actuator's driver part along the axis of displacement is well defined. Consequently, the actuator's driver part will generate an accurate force, both in terms of its magnitude and its rate of change.

In a first embodiment according to the invention, the actuator's support part is attached to the support by a support part bearing which has a support part bearing stiffness that provides a substantially zero displacement of the actuator's support part along the driver's axis of displacement during a displacement of the driver part along said axis with regard to the support part. Here, a support part bearing is to be interpreted as a device for connecting the actuator's support part to the support. Moreover, the support part bearing stiffness is the resistance of the support part bearing to deformation through an applied force or torque. The accuracy of needleless transdermal medication, which amounts to the forcible supply of medication through the skin within the context of this paper, will benefit from the support stiffness and the support bearing stiffness providing a substantially zero displacement of the actuator's support part along the driver's axis of displacement during a displacement of the driver part along said axis with regard to the support part. In this text, needleless transdermal delivery of medication amounts to the supply of medication in which the medication is forcibly introduced through the skin. The latter penetration of the skin is made possible by a substantially high velocity at which the medication approaches the skin. The magnitude and the rate of change for the force generated by the actuator are maximally transmitted owing to the support stiffness and the support bearing stiffness. Otherwise, the magnitude and the rate of change for pressure build up by way of the actuator would be absorbed by deformation of the support and the support part bearing.

In a second and preferred embodiment according to the invention, the support part bearing has a further support part bearing stiffness along an axis different from the axis of displacement. The further support part bearing stiffness provides a substantially zero displacement for the actuator's support part along the axis different from the axis of displacement during a displacement of the support along the axis different from the axis of displacement. Here, the support's displacement encompasses a deformation of the support. Furthermore, the support's displacement includes a deviation with respect to a support's nominal position with regard to the actuator's support part due to e.g. manufacturing tolerances. Through mechanically decoupling the support from the actuator's support part along the axis different from the driver's axis of displacement by way of the further support part bearing stiffness, an introduction of a potentially damaging mechanical stress into the actuator's support part along this axis is prevented from. This may be of particular relevance for e.g. actuators based on the piezo electric effect. Namely, these actuators comprise brittle ceramics. Even a limited stress along an axis different from a piezo electric element's direction of displacement, may cause irreversible damage to the piezo electric element which is a critical, and likely most expensive component of the device for needleless transdermal drug delivery.

In a third embodiment according to the invention, the support part bearing has a rotational support part bearing stiffness which provides a substantial zero rotation for the actuator's support part during a rotation of the support. Here the support's rotation includes a torsional deformation of the support. Moreover, in this text a rotation of the support encompasses a deviation with respect to a support's nominal angular orientation with regard to the actuator's support part due to e.g. manufacturing tolerances. As indicated before, through mechanically decoupling the support from the actuator's support part, the introduction of a potentially damaging mechanical stress into the actuator's support part is prevented from. Manufacturing tolerances eventuating in a deviation of the support's nominal position or nominal angular orientation with regard to the actuator's support part are a likely source for a potentially damaging mechanical stress. Consequently, by way of mechanically decoupling the support from the actuator's support part, the prerequisite for small manufacturing tolerances is circumvented.

In a fourth embodiment according to the invention, the support part bearing is implemented by an elastic support rod. Here elastic implies non-plastic. The elastic support rod has a length that is substantially larger than a dimension establishing an elastic rod's cross-section. As a result, the elastic support rod materializes a longitudinal stiffness which is substantially larger than a cross-sectional stiffness. The elastic support rod is an element of plain nature. Consequently, it enables a cost-limiting and robust implementation for the support bearing. A suitable material for support bearings is e.g. stainless steel.

In a fifth embodiment according to the invention, the support bearing is implemented by a support ball and socket joint. Such joints can be made from stainless steel. Furthermore, the actuator is pretensioned to the support ball and socket joint by a membrane which is elastically connected to the support. Owing to a ball's rotational degree of freedom, the ball and socket joint does not transmit a displacement along an axis perpendicular to the axis of displacement. In addition to that, the ball and socket joint does not transmit a rotation. The actuator is connected to the membrane by a driver part bearing. The driver part bearing has a driver part bearing stiffness which provides a substantially zero mutual displacement of the actuator's driver part and the membrane along the driver's axis of displacement during a displacement of the driver part along said axis with regard to the support part. As a result of this, the magnitude and the rate of change of the force generated by the actuator's driver part is maximally transmitted to the membrane rather than being absorbed.

In a sixth embodiment according to the invention, the driver part bearing has a further driver part bearing stiffness along an axis different from the axis of displacement. The further driver part bearing stiffness provides a substantially zero displacement for the actuator's driver part along the axis different from the driver's axis of displacement during a displacement of the membrane along said axis. Furthermore, the driver part bearing has a rotational driver part bearing stiffness which provides a substantially zero rotation for the actuator's driver part during a rotation of the membrane. As a result of this, the actuator is invariant under the support's and the membrane's displacement along an axis different from the axis of displacement as well as the support's and the membrane's rotation. More specifically, the features present in this embodiment decouple the actuator mechanically from its surroundings in the sense that no potentially damaging mechanical stress is introducible to the actuator along an axis different from the driver's axis of displacement. As a result, the sensitivity of the actuator for a potentially damaging stress along an axis different the axis of displacement is nullified. This quality is essential for the fruitful application of piezo electric elements. Thereby this embodiment allows for autonomous insertion of the cartridge by patients suffering from movement disorders such as Parkinson's disease.

In a seventh embodiment according to the invention, a reservoir for storing a fluid which is to be delivered transdermally, is connected to the support. The reservoir can comprise an orifice to deliver the fluid through. The latter design enables repetitive delivery of medication. For the purpose of possibly refilling the reservoir, the reservoir is connected or connectable to the support in a detachable manner. Alternatively, the reservoir may be implemented by a one shot design in which the force generated by the actuator's driver part effectuates a pressure in the reservoir such high that the reservoir detonates in a defined way and thereby delivers the medication through the skin.

In an eighth embodiment, the reservoir is a part of a disposable cartridge, more particularly a disposable cartridge. Alternatively, the cartridge is a refillable cartridge.

In a ninth embodiment according to the invention, the support comprises a sliding mechanism for inserting the cartridge into the support.

In a tenth and preferred embodiment according to the invention, the disposable cartridge has a slider for cooperation with the sliding mechanism incorporated in the support. With these features the operation of a system comprising the device and the disposable cartridge is largely facilitated. The latter can be elemental to e.g. patients suffering from Parkinson's disease. Preferably, the slider is provided with sliding rails or grooves for cooperation with corresponding grooves or rails of the sliding mechanism. Such sliding systems are known per se.

In an eleventh embodiment according to the invention the actuator is a piezo electric element. This type of actuator enables a large rate of change in the force generated by the driver part which is essential in needleless transdermal drug delivery. The piezo element may be a known element.

### BRIEF DESCRIPTION OF THE DRAWINGS

The invention and its advantages are further elucidated by way of example with reference to the drawings in which:

FIG. 1 schematically displays an embodiment of the device according to the invention wherein a piezoelectric actuator is attached to a support by way of an elastic rod and wherein a disposable cartridge is provided.

FIG. 2 schematically shows an embodiment of the device according to the invention wherein a piezoelectric actuator is attached to a support by a support ball and socket joint.

### DETAILED DESCRIPTION OF THE EMBODIMENTS

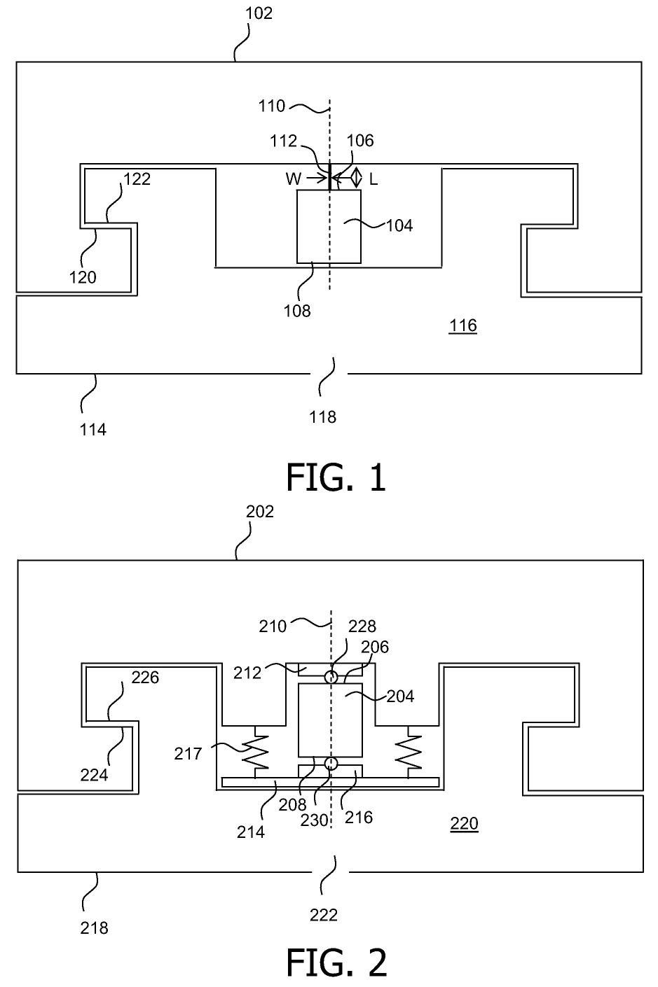

FIG. 1 displays a first and preferred embodiment according to the invention. The embodiment comprises a support 102 which has a support stiffness and which is stationary arranged. Furthermore, the embodiment comprises a piezo electric actuator 104 which has a support part 106 and a driver part 108 which is displaceable with regard to the support part 106 along an axis of displacement 110. The support part 106 is connected to the support 102 through a support bearing which has a support part bearing stiffness. The support part bearing comprises an elastic rod 112 made of stainless steel. The elastic rod 112 has a length L and a width W.

The support stiffness along with the support part bearing stiffness provide a substantially zero displacement of the support part 106 along the axis of displacement 110 during a displacement of the driver part 108 along the axis of displacement 110 with regard to the support part 106. Consequently, a displacement of the driver part 108 is well defined hence the driver part 108 will generate an accurate force, both in terms of its magnitude and its rate of change. For this purpose, the support 102 is made of titanium.

The support part bearing furthermore comprises a further support part bearing stiffness along an axis different from the axis of displacement. The further support part bearing stiffness provides a substantially zero displacement for the support part 106 along the axis different from the axis of displacement 110 during a displacement of the support 102 along the axis different from the axis of displacement 110. The support part bearing furthermore comprises a rotational support part bearing stiffness which provides a substantial zero rotation for the support part 106 during a rotation of the support 102. Namely, the elastic support rod 112 is aligned with the axis of displacement 110. In addition to that, the length L of the elastic rod 112 is substantially larger than the width W of the elastic rod 112. Consequently, the elastic rod 112 materializes a longitudinal stiffness which is substantially larger than a cross-sectional stiffness. As a result, the substantially zero displacement of the support part 106 along the axis of displacement 110 is provided during a displacement of the driver part 108 along the axis of displacement 110 with regard to the support part 106. Furthermore, the zero displacement is provided for the support part 106 along the axis different from the axis of displacement 110 during a displacement of the support 102 along the axis different form the axis of displacement 110. Additionally a zero rotation is provided for the support part 106 during a rotation of the support 102 by the elastic rod 112. As a result, an introduction of a potentially damaging mechanical stress into the support part 106 by way of a displacement or a rotation of the support 102 is prevented from. During use, the driver part 108 pressurizes upon activation of the actuator 104 a disposable cartridge 114 which stores a medication 116. The medication 116 is forced out of the cartridge through an orifice 118. The disposable cartridge 114 is inserted to the support by way of a sliding mechanism 120. The sliding mechanism 120 is attached to the support 102. For this purpose, the disposable cartridge 114 comprises a sliding rails 122 constituting a slider. The disposable cartridge 114 is movable into and out with regard to the support 102 in a direction perpendicular to the axis of displacement 110.

FIG. 2 depicts a second embodiment according to the invention. This embodiment comprises a support 202 which is stationary arranged and which support has a support stiffness. The embodiment furthermore comprises a piezo electric actuator 204 which has a support part 206 and a driver part 208 which is displaceable with regard to the support part 206 along an axis of displacement 210. The support part 206 is connected to the support 202 through a support bearing which has a support part bearing stiffness. The support part bearing comprises a support ball and socket joint 212 made of stainless steel. The piezo electric actuator 204 is pretensioned to the support 202 by a membrane 214 made of stainless steel. The membrane 214 is connected to the actuator 204 by means of a driver part bearing which has a driver part bearing stiffness. The driver part bearing comprises a driver ball and socket joint 216 made of stainless steel. The membrane 214 is furthermore attached to the support 202 through an elastic element 217 which comprises a mechanical spring.

The support stiffness along with the support part bearing stiffness provide a substantially zero displacement of the support part 206 along the axis of displacement 210 during a displacement of the driver part 208 along the axis of displacement 210 with regard to the support part 206. For this purpose, the support 202 is made of titanium. As a result of this, a displacement of the driver part 208 is well defined. Therefore the driver part 208 will generate an accurate force, both in terms of its magnitude and its rate of change.

Furthermore, the driver part bearing has a driver part bearing stiffness which provides a substantially zero mutual displacement of the driver part 208 and the membrane 214 along the axis of displacement 210 during a displacement of the driver part 208 along the axis of displacement 210 with regard to the support part 206. As a result, the magnitude and the rate of change of the force generated by the driver part 208 are maximally transmitted to the membrane 214.

The support part bearing furthermore comprises a further support part bearing stiffness along an axis different from the axis of displacement 210. The further support part bearing stiffness provides a substantially zero displacement for the support part 206 along the axis different from the axis of displacement 210 during a displacement of the support 202 along the axis different from the axis of displacement 210. The support part bearing furthermore comprises a rotational support part bearing stiffness which provides a substantial zero rotation for the support part 206 during a rotation of the support 202. Namely, the support part ball 228 contained in the support ball and socket joint 212 has both a translational degree of freedom along an axis different form the axis of displacement 210 and a rotational degree of freedom. Therefore, the support ball and socket joint 212 does not transmit a displacement along an axis perpendicular to the axis of displacement 210. In addition to that, the support ball and socket joint 212 does not transmit a rotation. As a result, the zero displacement is provided for the support part 206 along the axis different from the axis of displacement 210 during a displacement of the support 202 along the axis different from the axis of displacement 210. Additionally a zero rotation for the support part 206 during a rotation of the support 202 is provided. Consequently, the introduction of a potentially damaging mechanical stress into the support part 206 by way of a displacement of the support 202 along an axis different from the axis of displacement 210 or due to a rotation of the support 202, is prevented from.

The driver part bearing furthermore comprises a further driver part bearing stiffness along an axis different from the axis of displacement 210. The further driver part bearing stiffness provides a substantially zero displacement for the driver part 208 along the axis different from the axis of displacement 210 during a displacement of the membrane 214 along the axis different from the axis of displacement 210. The driver part bearing furthermore comprises a rotational driver part bearing stiffness which provides a substantial zero rotation for the driver part 208 during a rotation of the membrane 214. Namely, the driver part ball 230 contained in the driver ball and socket joint 216 has both a translational degree of freedom along an axis different form the axis of displacement 210 and a rotational degree of freedom. Therefore, the driver ball and socket joint 216 does not transmit a displacement along an axis perpendicular to the axis of displacement 210. In addition to that, the driver ball and socket joint 216 does not transmit a rotation. As a result, the zero displacement is provided for the driver part 208 along the axis different from the axis of displacement 210 during a displacement of the membrane 214 along the axis different form the axis of displacement 210. Additionally a zero rotation is provided for the driver part 208 during a rotation of the membrane 214. Consequently, the introduction of a potentially damaging mechanical stress into the driver part 208 by way of a displacement of the membrane 214 along an axis different from the axis of displacement 210 or due to a rotation of the membrane 214, is prevented from.

During use, the driver part 208 pressurizes a disposable cartridge 218 which stores a medication 220. The medication 220 is forced out of the cartridge through an orifice 222. The disposable cartridge 218 is inserted to the support by way of a sliding mechanism 224. For this purpose, the disposable cartridge 218 comprises a sliding rails 226.

While the invention has been illustrated and described in detail in the drawings and in the foregoing description, the illustrations and the description are to be considered illustrative or exemplary and not restrictive. The invention is not limited to the disclosed embodiments. For instance, the membrane and the elastic elements attaching the membrane to the support, may be substituted by a single elastic membrane unit. Furthermore, the support part ball and socket joint's position and the driver part ball and socket joint's position along the axis of displacement the membrane may be established by way of magnetic elements rather than by the pretensioned membrane.

It is noted that the apparatus according to the invention and all its components can be made by applying processes and materials known per se. In the set of claims and the description the word “comprising” does not exclude other elements and the indefinite article “a” or “an” does not exclude a plurality. Any reference signs in the claims should not be construed as limiting the scope. It is further noted that all possible combinations of features as defined in the set of claims are part of the invention.

1. A device for needleless transdermal delivery of medication, comprising an actuator and a support, which support has a support stiffness and which actuator has a driver part and a support part, which support part is connected to the support and which driver part is displaceable with regard to the support part along an axis of displacement, characterized in that the support stiffness provides a substantially zero displacement of the actuator's support part along the axis of displacement during a displacement of the driver part along said axis with regard to the support part.

2. The device according to claim 1 wherein the actuator's support part is connected to the support by a support part bearing which has a support part bearing stiffness which, along with the support stiffness, provides the substantially zero displacement of the actuator's support part along the axis of displacement during a displacement of the driver part along said axis with regard to the support part

3. The device according to claim 2 wherein the support part bearing has a further support part bearing stiffness along an axis different from the axis of displacement, which further support part bearing stiffness provides a substantially zero displacement for the actuator's support part along the axis different from the axis of displacement during a displacement of the support along the axis different from the axis of displacement.

4. The device according to claim 2 wherein the support part bearing has a rotational support part bearing stiffness which provides a substantially zero rotation for the actuator's support part during a rotation of the support.

5. The device according to claim 2 wherein the support part bearing comprises an elastic support rod which has a length that is substantially larger than a width of an elastic rod's cross-section.

6. The device according to claim 2, 3 or 4 wherein the support part bearing is a support part ball and socket joint to which the actuator is pretensioned by a membrane, which membrane is connected to the actuator's driver part through a driver part bearing, which has a driver part bearing stiffness that provides a substantially zero mutual displacement of the actuator's driver part and the membrane along the axis of displacement during a displacement of the driver part along said axis with regard to the support part.

7. The device according to claim 6 wherein the driver part bearing has a further driver part bearing stiffness along an axis different from the axis of displacement, which further driver part bearing stiffness provides a substantially zero displacement for the actuator's driver part along the axis different from the axis of displacement during a displacement of the membrane along the axis different from the axis of displacement, and which driver part bearing has a rotational driver part bearing stiffness which provides a substantially zero rotation for the actuator's driver part during a rotation of the membrane.

8. The device according to claim 6 wherein the driver part bearing comprises a driver part ball and socket joint.

9. The device according to claim 1 wherein a reservoir for storing a fluid is detachably connectable to the support, which reservoir is pressurizable by the actuator's driver part.

10. The device according to claim 9 wherein the reservoir is part of a disposable cartridge.

11. The device according to claim 10 wherein the disposable cartridge is inserted into the support through a sliding mechanism of the support.

12. The device according to claim 10 wherein the cartridge has a slider for cooperation with the sliding mechanism of the support.

13. The device according to claim 1 wherein the actuator is a piezo electric element.

14. A cartridge for use in a device according to claim 12 and provided with a slider for cooperation with the sliding mechanism of the device.
